(19) United States
(12) Patent Application Publication
(10) Pub. No.: US 2011/0166566 A1
(43) Pub. Date: Jul. 7, 2011

(54) DEVICES AND METHODS FOR PERFORMING PERCUTANEOUS SURGICAL PROCEDURES

(75) Inventor: Simon Gabriel, Redlands, CA (US)

(73) Assignee: LOMA LINDA UNIVERSITY MEDICAL CENTER, Loma Linda, CA (US)

(21) Appl. No.: 12/991,852

(22) PCT Filed: Jul. 1, 2010
(86) PCT No.: PCT/US10/40840

§ 371 Date: Nov. 9, 2010

Related U.S. Application Data
(60) Provisional application No. 61/222,672, filed on Jul. 2, 2009.
(60) Provisional application No. 61/227,707, filed on Jul. 22, 2009.
(60) Provisional application No. 61/234,569, filed on Aug. 17, 2009.

(51) Int. Cl. A61B 18/00; A61M 25/00

(52) U.S. Cl. 606/34; 604/528

(57) ABSTRACT

A guidewire or a catheter with a stiffness that can be varied during use inside a human body, where the guidewire or the catheter comprises a magnetorheological fluid. A guidewire comprising a proximal section, a distal section, and an intermediate section connecting the proximal section and the distal section, where the intermediate section comprises a longitudinal axis, and the distal section comprises a longitudinal axis, and where the longitudinal axis of the distal section can be coincident with the longitudinal axis of the intermediate section or can be controllably made non-coincident with the longitudinal axis of the intermediate section during use inside a human body, and the guidewire further comprises a plurality of piezoelectric cores and one or more than one piezoelectric strut.

### CROSS-REFERENCE TO RELATED APPLICATIONS

The present Application claims the benefit of United States Provisional Patent Application No. 61/222,672 entitled “Variable Stiffness Surgical Catheter,” filed Jul. 2, 2009; United States Provisional Patent Application No. 61/227,707 entitled “Variable Stiffness Surgical Guidewire and Catheter,” filed Jul. 22, 2009; and United States Provisional Patent Application No. 61/234,569 entitled “Devices and Methods for Performing Percutaneous Surgical Procedures,” filed Aug. 17, 2009; the contents of which are incorporated in this disclosure by reference in their entirety.

### BACKGROUND

Percutaneous surgical procedures are used to treat a variety of diseases and conditions as an alternate to standard open procedures. Among the diseases and conditions is coronary artery disease caused by an intraarterial plaque narrowing the lumen of a coronary artery which is frequently treated by a percutaneous endovascular procedure. The percutaneous endovascular treatment of coronary artery disease (percutaneous transluminal angioplasty) involves accessing an artery by a hollow needle, inserting a guidewire through the hollow needle and into the artery, and removing the needle. Then, the guidewire is advanced until the distal end of the guidewire traverses or is positioned near the intra-arterial plaque. Next, a catheter with a central lumen is threaded over the guidewire, and traverses or is positioned near the intra-arterial plaque and the guidewire is removed. Then, a treatment device is passed through the central lumen of the catheter and the intra-arterial plaque is treated. Finally, the treatment device and catheter are removed.

In order to position the guidewire and catheter properly for percutaneous transluminal angioplasty, the guidewire and the catheter must follow the curves of the arterial lumen in which the guidewire and the catheter are placed. To meet this requirement, guidewires and catheters are available in a variety of lengths, materials, stiffnesses, thicknesses, and tip configurations. However, even with all of the various guidewires and catheters available, there are still many situations where the procedure cannot be completed because the guidewires and catheters cannot negotiate the curves of the arterial lumen.

Therefore, there is a need for a guidewire that can better negotiate the curves of the arterial lumen during an endovascular procedure than standard guidewires. Further, there is a need for a catheter that can better negotiate the curves of the arterial lumen during an endovascular procedure than standard catheters. Further, there is a need for an improved method of performing a procedure within a human body, such as for example percutaneous transluminal angioplasty that is not associated with the disadvantages of present methods.

### SUMMARY

According to one embodiment of the present invention, there is provided a guidewire with a stiffness that can be varied during use. The guidewire comprises a) a proximal end and a distal end, and oriented from the proximal end to the distal end, a proximal section, an intermediate section and a distal section, where the intermediate section connects the proximal section to the distal section; b) an outer layer comprising and defined by an outer surface of the outer layer and an opposing inner surface of the outer layer; c) a central fluid layer defined by the inner surface of the outer layer, and surrounded by the outer layer; and d) a longitudinal length between the proximal end and a distal end; e) a cross-sectional area defined by the outer surface of the outer layer; where the outer layer further comprises one or more than one circumferential wire comprising a material that generates a magnetic field in response to the application of electric current; where the central fluid layer comprises magnetorheological fluid comprising a suspension of micrometer-sized magnetic particles suspended in a carrier fluid; and where the central fluid layer further comprises a viscosity. In one embodiment, the longitudinal length is between 50 cm and 250 cm. In another embodiment, the longitudinal length is between 100 cm and 200 cm. In another embodiment, the cross-sectional area is between 1.0 mm² and 4.0 mm² In another embodiment, the cross-sectional diameter is between 1.5 mm² and 3.0 mm² In another embodiment, the proximal section of the guidewire comprises a proximal seal for sealing the central fluid layer at the proximal end of the guidewire, and the distal section of the guidewire comprises a distal seal for sealing the central fluid layer at the distal end of the guidewire. In another embodiment, the one or more than one circumferential wire is a plurality of circumferential wires. In another embodiment, the one or more than one circumferential wire is oriented in a circumferential spiral around the central fluid layer. In another embodiment, the one or more than one circumferential wire comprises copper or iron. In another embodiment, the one or more than one circumferential wire comprises insulated copper or insulated iron. In another embodiment, the outer layer further comprises one or more than one accessory wire encircling each of the one or more than one circumferential wire. In another embodiment, the one or more than one accessory wire comprises copper. In another embodiment, the one or more than one circumferential wire comprises iron and the one or more than one accessory wire comprises copper. In another embodiment, the outer layer further comprises a plurality of longitudinally directed ribs comprising a material that generates a magnetic field in response to the application of electric current. In another embodiment, the ribs comprise copper or iron. In another embodiment, the ribs comprise insulated copper or insulated iron. In another embodiment, the outer layer further comprises between two ribs and ten ribs. In another embodiment, the outer layer further comprises between two ribs and six ribs. In another embodiment, the outer layer further comprises four ribs. In another embodiment, the outer layer further comprises a biocompatible material. In another embodiment, the biocompatible material is selected from the group consisting of an epoxy resin and a polyurethane resin. In another embodiment, the carrier fluid is selected from the group consisting of mineral oil, polyethelene glycol, a synthetic oil and water. In another embodiment, the carrier fluid is silicon oil. In another embodiment, the magnetic particles comprise ferrometallic particles. In another embodiment, the magnetorheological fluid further comprises a surfactant. In another embodiment, the surfactant is selected from the group consisting of citric acid, oleic acid, and soy lecithin. In another embodiment, the guidewire further comprises a plurality of segments, where each segment comprises a separate circumferential wire or a plurality of separate circumferential wires. In one embodiment, the plurality of segments is three segments. In another embodiment, the plurality of segments comprises more than three segments. In another embodiment, the plurality of segments comprises five or more than five segments. In another embodiment, the plurality of segments comprises ten or more than ten segments. In another embodiment, only the proximal section of the guidewire comprises a plurality of segments. In another embodiment, only the intermediate section of the guidewire comprises a plurality of segments. In another embodiment, only the distal section of the guidewire comprises a plurality of segments. In another embodiment, both the proximal section and the intermediate section comprise a plurality of segments. In another embodiment, both the intermediate section and the distal section comprise a plurality of segments. In another embodiment, both the proximal section and the distal section comprise a plurality of segments. In another embodiment, the proximal section, the intermediate section and the distal section comprise a plurality of segments. In another embodiment, each segment of the plurality of segments further comprises an electronic gate. In another embodiment, the electronic gate is a field effect transistor located between adjoining segments of the plurality of segments, and each segment further comprises a source wire connected to each electronic gate; and where the guidewire further comprises one or more than one gating wire connected to each electronic gate, and a drain wire connected to all of the circumferential wires. In another embodiment, each segment of the plurality of segments is separated from an adjoining segment by a barrier; and where each barrier comprises a material that prevents the magnetorheological fluid in one segment from communicating with the magnetorheological fluid in the adjoining segment. In another embodiment, the barrier comprises polyurethane. According to another embodiment of the present invention, there is provided a system for performing a procedure within a human body. The system comprises a) one or more than one guidewire according to the present invention; and b) a control for controllably sending electric current through the one or more than one circumferential wire; where the control is connected to the guidewire through the proximal section of the guidewire. According to another embodiment of the present invention, there is provided a method for performing a procedure within a human body. The method comprises a) providing a guidewire according to the present invention; and b) using the guidewire to perform the procedure. According to another embodiment of the present invention, there is provided a method for performing a procedure within a human body. The method comprises a) providing a system according to the present invention; and b) generating electric current through the control and sending electric current through the one or more than one circumferential wire, thereby creating a magnetic flux in the fluid layer causing the magnetic particles suspended in the carrier fluid to become aligned which increases the viscosity of the fluid by decreasing the ability of the carrier fluid to flow around the particles, thereby controllably increasing the stiffness of the guidewire.

According to another embodiment of the present invention, there is provided a catheter with a stiffness that can be varied during use inside a human body, the catheter comprises a) a proximal end and a distal end, and oriented from the proximal end to the distal end, a proximal section, an intermediate section and a distal section, where the intermediate section connects the proximal section to the distal section; b) an outer layer comprising and defined by an outer surface of the outer layer, and an opposing inner surface of the outer layer; c) an inner layer comprising and defined by an outer surface of the inner layer, and an opposing inner surface of the inner layer; d) a fluid layer between and defined by the inner surface of the outer layer, and the outer surface of the inner layer; e) a central lumen defined by the inner surface of the inner layer; f) a longitudinal length between the proximal end and the distal end; and g) a cross-sectional area defined by the outer surface of the outer layer; where the outer layer of the catheter comprises and is defined by an outer surface of the outer layer; where the outer layer further comprises one or more than one circumferential wire comprising a material that generates a magnetic field in response to the application of electric current; where the fluid layer comprises magnetorheological fluid comprising a suspension of micrometer-sized magnetic particles suspended in a carrier fluid; and where the magnetorheological fluid comprises a viscosity. In one embodiment, the longitudinal length is between 50 cm and 250 cm. In another embodiment, the longitudinal length is between 100 cm and 200 cm. In another embodiment, the cross-sectional area is between 0.2 mm² and 7 mm² In another embodiment, the cross-sectional area is between 0.2 mm² and 3.5 mm² In another embodiment, the proximal section comprises a proximal seal for sealing the fluid layer at the proximal end of the catheter. In another embodiment, the proximal seal is configured to connect to a surgical instrument once the surgical instrument is inserted into the central lumen of the catheter. In another embodiment, the distal section of the catheter comprises a distal seal for sealing the fluid layer at the distal end of the catheter. In another embodiment, the one or more than one circumferential wire is a plurality of circumferential wires. In another embodiment, the one or more than one circumferential wire is oriented in a circumferential spiral around the fluid layer. In another embodiment, the one or more than one circumferential wire comprises copper or iron. In another embodiment, the one or more than one circumferential wire comprises insulated copper or insulated iron. In another embodiment, the outer layer further comprises one or more than one accessory wire encircling each of the one or more than one circumferential wire. In another embodiment, the one or more than one accessory wire comprises copper. In another embodiment, the one or more than one circumferential wire comprises iron and the one or more than one accessory wire comprises copper. In another embodiment, the outer layer further comprises a plurality of longitudinally directed ribs comprising a material that generates a magnetic field in response to the application of electric current. In another embodiment, the ribs comprise copper or iron. In another embodiment, the ribs comprise insulated copper or insulated iron. In another embodiment, the outer layer further comprises between four ribs and twenty ribs. In another embodiment, the outer layer further comprises between six ribs and fifteen ribs. In another embodiment, the outer layer comprises twelve ribs. In another embodiment, the outer layer comprises a biocompatible material selected from the group consisting of an epoxy resin and a polyurethane resin. In another embodiment, the inner layer comprises a biocompatible material selected from the group consisting of an epoxy resin and a polyurethane resin. In another embodiment, the inner surface of the inner layer further comprises a lubricious coating adjacent the central lumen to decrease frictional resistance to guidewires or other devices passing through the central lumen. In another embodiment, the one or more than one circumferential wire is a plurality of wires. In another embodiment, the one or more than one circumferential wire is oriented in a circumferential spiral between the fluid layer and the central lumen. In another embodiment, the one or more than one circumferential wire comprises copper or iron. In another embodiment, the one or more than one circumferential wire comprises insulated copper or insulated iron. In another embodiment, the inner layer further comprises one or more than one accessory wire encircling each of the one or more than one circumferential wire. In another embodiment, the one or more than one accessory wire comprises copper. In another embodiment, the magnetorheological fluid is selected from the group consisting of mineral oil, polyethelene glycol, a synthetic oil and water. In another embodiment, the magnetorheological fluid is silicon oil. In another embodiment, the magnetic particles comprise ferrometallic particles. In another embodiment, the magnetorheological fluid further comprises a surfactant to offset the inherent density difference between the magnetic particles and the carrier fluid. In one embodiment, the surfactant is selected from the group consisting of citric acid, oleic acid, and soy lecithin. In another embodiment, the central lumen comprises a cross-sectional area defined by the inner surface of the inner layer, and where the cross-sectional area of the central lumen is between 1.2 mm² and 4.2 mm² In another embodiment, the central lumen comprises a cross-sectional area defined by the inner surface of the inner layer, and where the cross-sectional area of the central lumen is between 1.7 mm² and 3.2 mm² In another embodiment, the catheter further comprises a plurality of segments, and each segment comprises a separate circumferential wire in the outer layer or comprises a plurality of circumferential wires in the outer layer that are separate from the other segments. In another embodiment, the plurality of segments comprises three segments. In another embodiment, the plurality of segments comprises more than three segments. In another embodiment, the plurality of segments comprises five or more than five segments. In another embodiment, the plurality of segments comprises ten or more than ten segments. In another embodiment, the catheter further comprises a corresponding accessory wire or corresponding accessory wires for each circumferential wire. In another embodiment, only the proximal section of the catheter comprises a plurality of segments. In another embodiment, only the intermediate section of the catheter comprises a plurality of segments. In another embodiment, only the distal section of the catheter comprises a plurality of segments. In another embodiment, both the proximal section and the intermediate section comprise a plurality of segments. In another embodiment, both the intermediate section and the distal section comprise a plurality of segments. In another embodiment, both the proximal section and the distal section comprise a plurality of segments. In another embodiment, each of the proximal section, the intermediate section and the distal section comprise a plurality of segments. In another embodiment, where each segment of the plurality of segments further comprises an electronic gate. In another embodiment, the electronic gate is a field effect transistor (FET) located between adjoining segments, and where the plurality of segments further comprise a source wire connected to each electronic gate and a drain wire connected to all of the circumferential wires. In another embodiment, each segment is separated from an adjoining segment by a barrier; and where each barrier comprises a material that prevents the magnetorheological fluid in one segment from communicating with the magnetorheological fluid in the adjoining segment. In another embodiment, the barrier comprises polyurethane. According to another embodiment of the present invention, there is provided a system for performing a procedure within a human body. The system comprises a) one or more than one catheter according to the present invention; and b) a control for controllably sending electric current through the one or more than one circumferential wire; where the control is connected to the catheter through the proximal section of the catheter. In another embodiment, there is provided a method for performing a procedure within a human body. The method comprises a) providing a catheter according to the present invention; and b) using the catheter to perform the procedure. According to another embodiment, there is provided a method for performing a procedure within a human body. The method comprises a) providing a system according to the present invention; and b) generating electric current through the control and sending electric current through the one or more than one circumferential wire, thereby creating a magnetic flux in the fluid layer causing the magnetic particles suspended in the carrier fluid to become aligned which increases the viscosity of the magnetorheological fluid in one or more than one segment and therefore the stiffness in one or more than one segment.

According to another embodiment of the present invention, there is provided a guidewire that can be controllably bent during use, where the guidewire comprises a plurality of piezoelectric struts comprising a piezoelectric material. According to another embodiment of the present invention, there is provided a guidewire than can controllably bend during use. The guidewire comprises a) a proximal end and a distal end, and oriented from the proximal end to the distal end, a proximal section, an intermediate section and a distal section, where the intermediate section connects the proximal section to the distal section; b) a longitudinal axis of the proximal section, a longitudinal axis of the intermediate section, and a longitudinal axis of the distal section, where the longitudinal axis of the intermediate section connects the longitudinal axis of the proximal section to the longitudinal axis of the distal section, and where the longitudinal axis of the proximal section, the longitudinal axis of the intermediate section, and the longitudinal axis of the distal section define a longitudinal axis of the guidewire; c) a longitudinal length between the proximal end and the distal end; d) one or more than one outer layer comprising and defined by an outer surface of the outer layer, and an opposing inner surface of the outer layer; e) a central layer defined by the inner surface of the outer layer, and surrounded by the outer layer; f) a cross-sectional area defined by the outer surface of the outer layer; g) one or more than one actuator base, each actuator base comprising a base plate, a first piezoelectric core, a second piezoelectric core, a first brace, a second brace, and a pivot connecting the first brace to the second brace; and h) one piezoelectric strut comprising a piezoelectric material associated with each actuator base; where the base plate spans the central layer between the inner surface of the outer layer and is attached to two opposing locations of the inner surface of the outer layer; where the first piezoelectric core is adjacent the base plate, and extends across the central layer between the inner surface of the outer layer without contacting the inner surface of the outer layer at any location; where the second piezoelectric core extends across the central layer between the inner surface of the outer layer without contacting the inner surface of the outer layer at any location; where the first piezoelectric core comprises a long axis, and the second piezoelectric core comprises a long axis; where the second piezoelectric core is spaced apart from the first piezoelectric core at a distance x, and the long axis of the first piezoelectric core is parallel to the long axis of the second piezoelectric core; where the first piezoelectric core and the second piezoelectric core comprise a piezoelectric material; where the first brace comprises a proximal end and a distal end; where the second brace comprises a proximal end and a distal end; where the first brace comprises a first casing at the proximal end of the first brace and a second casing at the distal end of the first brace; where the second brace comprises a first casing at the proximal end of the second brace and a second casing at the distal end of the second brace; where the first casing of the first brace and the first casing of the second brace surround opposing ends of the first piezoelectric core; where the second casing of the first brace and the second casing of the second brace surround opposing ends of the second piezoelectric core; where the first casing of the first brace comprises a first portion of a first connector and the base plate comprises a second portion of a first connector; where the first casing of the second brace comprises a first portion of a second connector and the base plate comprises a second portion of a second connector; where the first connector and the second connector allow the first piezoelectric core to expand longitudinally thereby increasing longitudinal dimension toward the inner surface of the outer layer while maintaining a relative distance to the base plate; where the piezoelectric strut comprises a proximal end and a distal end; where the proximal end of the piezoelectric strut is connected to either the second casing of the first brace or the second casing of the second brace; and where the distal end of the piezoelectric strut is connected to the inner surface of the outer layer in the distal section of the guidewire. In one embodiment, the longitudinal length of the guidewire is between 50 cm and 250 cm. In another embodiment, the longitudinal length of the guidewire is between 100 cm and 200 cm. In another embodiment, the cross-sectional area is between 0.4 mm² and 4.0 mm² In another embodiment, the cross-sectional diameter is between 0.4 mm² and 1.0 mm² In another embodiment, the distal section of the guidewire is tapered at the distal end of the guidewire. In another embodiment, the outer layer comprises a biocompatible material selected from the group consisting of an epoxy resin and a polyurethane resin. In another embodiment, the base plate comprises polyurethane or an epoxy resin composite. In another embodiment, the first portion of the first connector and the first portion of the second connector are male type extensions, and the second portion of the first connector and the second portion of the second connector are female type portions such as slotted grooves which mate with the male type extensions. In another embodiment, the guidewire further comprises micrometer sized ball bearings between the first portion of the first connector and the second portion of the first connector, or between the first portion of the second connector and the second portion of the second connector to decrease friction. In another embodiment, the first portion of the first connector and the second portion of the first connector, or the first portion of the second connector and the second portion of the second connector comprise a coating to decrease friction. In another embodiment, the first brace and the second brace comprise iron or aluminum In another embodiment, the first brace and the second brace comprise hardened polyurethane. In another embodiment, the first casing of the first brace, the second casing of the first brace, the first casing of the second brace and the second casing of the second brace each comprise a plastic such as hardened polyurethane. In another embodiment, the pivot is a screw comprising a head at one end and a distal end extending through matching holes in the center of the first brace and the center of the second brace, where the pivot comprises a transverse hole in the distal end to receive a pin, thereby fixing the pivot in place with respect to the first brace and the second brace. In another embodiment, the guidewire comprises one actuator base and associated piezoelectric strut. In another embodiment, the guidewire comprises a plurality of actuator bases and associated piezoelectric struts. In another embodiment, the guidewire comprises two actuator bases and associated piezoelectric struts. In another embodiment, the guidewire comprises three actuator bases and associated piezoelectric struts. In another embodiment, the guidewire comprises four actuator bases and associated piezoelectric struts. In another embodiment, the guidewire comprises more than four actuator bases and associated piezoelectric struts. In another embodiment, the guidewire comprises a plurality of actuator bases, each actuator base associated with one piezoelectric strut; and where the actuator bases are spaced apart from each other longitudinally, and are rotated relative to one another axially, thereby causing the distal end of each piezoelectric strut to be connected to the inner surface of the outer layer in the distal section of the guidewire at a different location. According to another embodiment of the present invention, there is provided a system for performing a procedure within a human body. The system comprises a) one or more than one guidewire according to the present invention; and b) a control connected to the guidewire for controlling the function of the guidewire. In one embodiment, the guidewire in the system further comprises wiring connecting the first piezoelectric core and the second piezoelectric core to the control for controlling transmission of current from the control to the first piezoelectric core and the second piezoelectric core. In another embodiment, the guidewire in the system further comprises wiring connecting the piezoelectric strut to the control for controlling transmission of current from the control to the piezoelectric strut. In another embodiment, the guidewire in the system further comprises separate wiring connecting the first piezoelectric core and the second piezoelectric core of each actuator base to the control for controlling transmission of current from the control to the first piezoelectric core and the second piezoelectric core. In another embodiment, the guidewire in the system further comprises separate wiring connecting the piezoelectric strut associated with each actuator base to the control for controlling transmission of current from the control to the piezoelectric strut. According to another embodiment of the present invention, there is provided a method for performing a procedure within a human body. In one embodiment, the method comprises a) providing a guidewire according to the present invention; and b) using the guidewire to perform the procedure. In another embodiment, the method comprises a) providing a system according to the present invention; and b) using the system to perform the procedure. In another embodiment, the method comprises a) providing a guidewire according to the present invention; b) inserting the guidewire in a first configuration into an arterial lumen of the patient percutaneously; c) supplying current to the one or more than one piezoelectric strut rendering the one or more than one piezoelectric strut in a first longitudinal length, and no current is being supplied to the one or more than one first piezoelectric core and the second piezoelectric core rendering them in a first longitudinal length, where the longitudinal axis of the distal section of the guidewire is coincident with the longitudinal axis of the intermediate section of the guidewire; and d) in order to negotiate a curve of the arterial lumen, changing the guidewire into a second configuration by supplying current to the wiring to the one or more than one first piezoelectric core and the second piezoelectric core rendering them in a second longitudinal length and increasing the distance x, the axial separation of first piezoelectric core and the second piezoelectric core and decreasing the separation of the first casing of the first brace from the first casing of the second brace, and the separation of the second casing of the first brace from the second casing of the second brace, and by simultaneously ceasing to supply current to the wiring to the one or more than one piezoelectric strut rendering the one or more than one piezoelectric strut in a second longitudinal length; where the first longitudinal length of the one or more than one first piezoelectric core and the second piezoelectric core is longer than the second longitudinal length of the one or more than one first piezoelectric core and the second piezoelectric core; and where the first longitudinal length of the one or more than one piezoelectric strut is shorter than the second longitudinal length of the one or more than piezoelectric strut, thereby causing the longitudinal axis of the distal section of the guidewire to become non-coincident with the longitudinal axis of the intermediate section of the guidewire. In one embodiment, the first longitudinal length of the one or more than one first piezoelectric core and the second piezoelectric core is longer than the second longitudinal length of the one or more than one first piezoelectric core and the second piezoelectric core by at least 25%. In another embodiment, the first longitudinal length of the one or more than one first piezoelectric core and the second piezoelectric core is longer than the second longitudinal length of the one or more than one first piezoelectric core and the second piezoelectric core by at least 50%. In another embodiment, the first longitudinal length of the one or more than one first piezoelectric core and the second piezoelectric core is longer than the second longitudinal length of the one or more than one first piezoelectric core and the second piezoelectric core by at least 100%. In another embodiment, the second longitudinal length of the one or more than one piezoelectric strut is longer than the first longitudinal length of the one or more than piezoelectric strut by at least 25%. In another embodiment, the second longitudinal length of the one or more than one piezoelectric strut is longer than the first longitudinal length of the one or more than piezoelectric strut by at least 50%. In another embodiment, the second longitudinal length of the one or more than one piezoelectric strut is longer than the first longitudinal length of the one or more than piezoelectric strut by at least 100%.

### FIGURES

These and other features, aspects and advantages of the present invention will become better understood with regard to the following description, appended claims, and accompanying figures where:

FIG. 1 is a partial, lateral perspective view of one embodiment of a system according to the present invention for performing a procedure within a human body, where the system comprises a guidewire according to the present invention;

FIG. 2 is a partial, close-up longitudinal cutaway view of one embodiment of the guidewire shown in FIG. 1 taken along the line 2-2;

FIG. 3 is a close-up cross-sectional view of one embodiment of the guidewire shown in FIG. 1 taken along the line 3-3;

FIG. 4 is a partial, close-up longitudinal cutaway view of another embodiment of the guidewire shown in FIG. 1 taken along the line 4-4;

FIG. 5 is a partial, lateral perspective view of one embodiment of a system according to the present invention for performing a procedure within a human body, where the system comprises a catheter according to the present invention;

FIG. 6 is a partial, close-up longitudinal cutaway view of one embodiment of the catheter shown in FIG. 5 taken along the line 6-6;

FIG. 7 is a cross-sectional view of one embodiment of the catheter shown in FIG. 5 taken along the line 7-7;

FIG. 8 is a partial, close-up longitudinal cutaway view of another embodiment of the catheter shown in FIG. 5 taken along the line 8-8;

FIG. 9 is a partial, lateral perspective view of one embodiment of a system according to the present invention for performing a procedure within a human body, where the system comprises a guidewire according to the present invention comprising a plurality of piezoelectric elements;

FIG. 10 is a partial, close-up longitudinal cutaway view of one embodiment of the guidewire shown in FIG. 9 taken along the line 10-10;

FIG. 11 is another a partial, close-up longitudinal cutaway view of one embodiment of the guidewire shown in FIG. 9 and FIG. 10 taken along the line 11-11; and

FIG. 12 is a cross-sectional view of one embodiment of a guidewire according to the present invention, taken along the line 12-12.

### DESCRIPTION

According to one embodiment of the present invention, there are provided devices that can better negotiate the curves of the arterial lumen during an endovascular procedure than standard guidewires and standard catheters. In one embodiment, the device is a guidewire comprising a stiffness that can be varied during use inside a human body, and the guidewire further comprises a magnetorheological fluid. In another embodiment, the device is a catheter comprising a stiffness that can be varied during use inside a human body, and the catheter further comprises a magnetorheological fluid. In another embodiment, the device is a guidewire comprising a proximal section, a distal section, and an intermediate section connecting the proximal section and the distal section, where the intermediate section comprises a longitudinal axis, and the distal section comprises a longitudinal axis, and where the longitudinal axis of the distal section can be coincident with the longitudinal axis of the intermediate section or can be controllably made non-coincident with the longitudinal axis of the intermediate section during use inside a human body, and the guidewire further comprises a plurality of piezoelectric cores and one or more than one piezoelectric strut. According to another embodiment of the present invention, there is provided a system for performing a procedure within a human body, such as for example percutaneous transluminal angioplasty. The system comprises one or more than one device according to the present invention, such as for example one or more than one guidewire according to the present invention, one or more than one catheter according to the present invention, or both one or more than one guidewire according to the present invention and one or more than one catheter according to the present invention. According to the present invention, there is provided a method for performing a procedure within a human body, such as for example percutaneous transluminal angioplasty. In one embodiment, the method comprises providing a device according to the present invention, and using the device to perform the procedure, where the device is one or more than one guidewire according to the present invention, one or more than one catheter according to the present invention, or both one or more than one guidewire according to the present invention and one or more than one catheter according to the present invention. In another embodiment, the method comprises providing a system according to the present invention and using the system to perform the procedure. The device, system and the method will now be disclosed in detail.

As used herein, except where context requires otherwise, the term “comprise” and variations of the term, such as “comprising,” “comprises,” and “comprised” are not intended to exclude other additives, components, integers, or steps.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

As used in this disclosure, except where the context requires otherwise, the method steps disclosed and shown are not intended to be limiting nor are they intended to indicate that each step is essential to the method or that each step must occur in the order disclosed.

The guidewire, catheter and system can be made according to techniques known to those with skill in the art, as will be understood by those with skill in the art with reference to this disclosure.

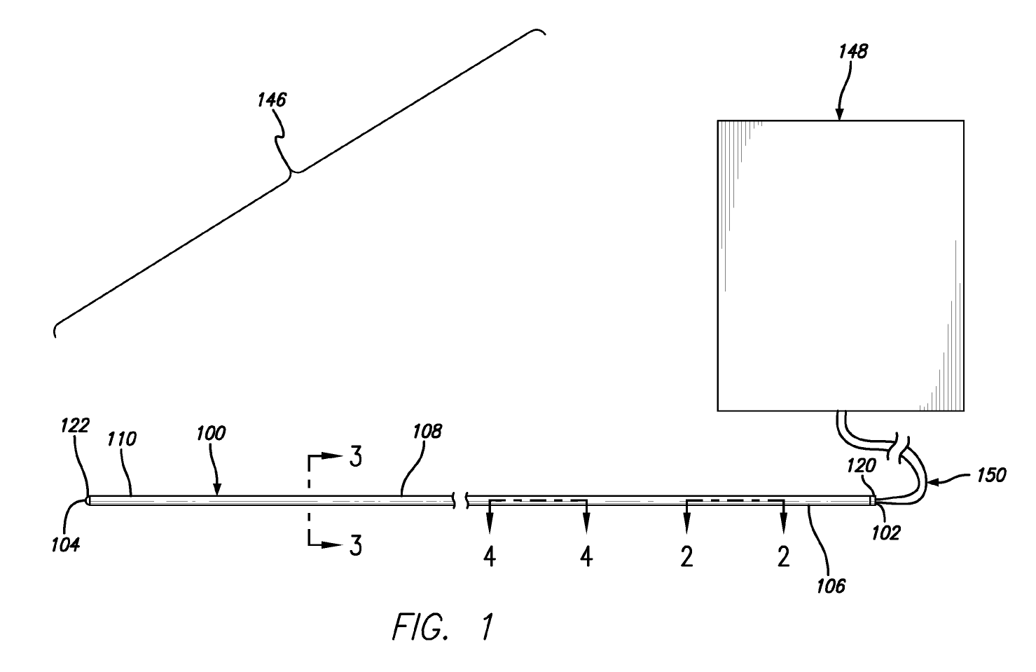

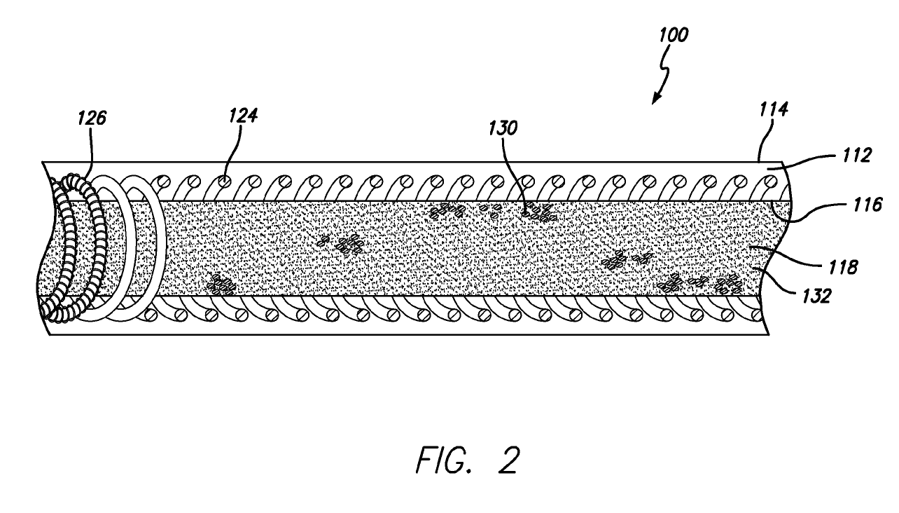

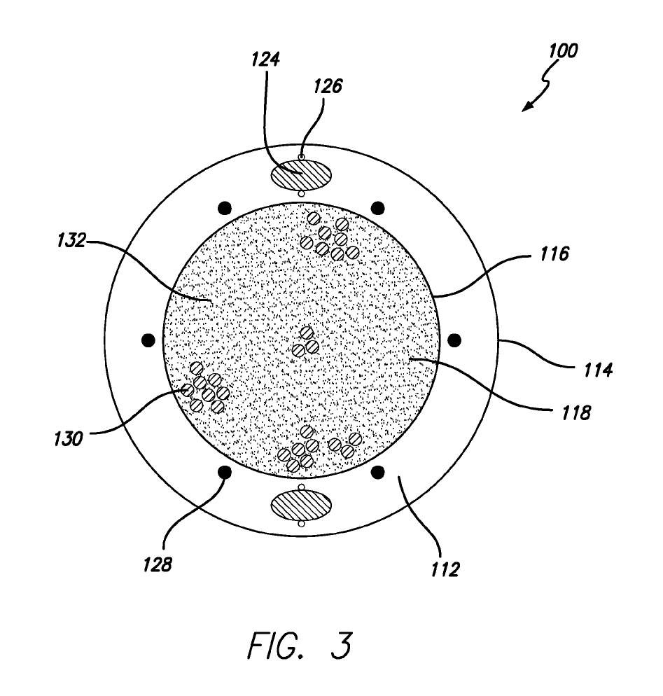

According to one embodiment of the present invention, there is provided a guidewire with a stiffness that can be varied during use inside a human body. The guidewire comprises a magnetorheological fluid. Referring now to FIG. 1, FIG. 2 and FIG. 3, there are shown, respectively, a partial, lateral perspective view of one embodiment of a system according to the present invention for performing a procedure within a human body, where the system comprises a guidewire according to the present invention (FIG. 1); a partial, close-up longitudinal cutaway view of one embodiment of the guidewire shown in FIG. 1 taken along the line 2-2 (FIG. 2); and a close-up cross-sectional view of one embodiment of the guidewire shown in FIG. 1 taken along the lines 3-3 (FIG. 3). As can be seen, the guidewire 100 comprises a proximal end 102 and a distal end 104, and further comprises, oriented from the proximal end 102 to the distal end 104, a proximal section 106, an intermediate section 108 and a distal section 110, where the intermediate section 108 connects the proximal section 106 to the distal section 110. The guidewire 100 further comprises an outer layer 112 comprising and defined by an outer surface 114 of the outer layer 112 and an opposing inner surface 116 of the outer layer 112. The guidewire 100 further comprises a central fluid layer 118 defined by the inner surface 116 of the outer layer 112, and surrounded by the outer layer 112. The guidewire 100 further comprises a longitudinal length between the proximal end 102 and a distal end 104. In one embodiment, the longitudinal length is between 50 cm and 250 cm. In another embodiment, the longitudinal length is between 100 cm and 200 cm. The guidewire 100 further comprises a cross-sectional area defined by the outer surface 114 of the outer layer 112. In one embodiment, the cross-sectional area is between 1.0 mm² and 4.0 mm² In another embodiment, the cross-sectional diameter is between 1.5 mm² and 3.0 mm²

The proximal section 106 of the guidewire 100 comprises a proximal seal 120 for sealing the central fluid layer 118 at the proximal end 102 of the guidewire 100 and the distal section 110 of the guidewire 100 comprises a distal seal 122 for sealing the central fluid layer 118 at the distal end 104 of the guidewire 100. In one embodiment, the distal end 104 of the guidewire 100 is tapered as shown in FIG. 1 forming the distal seal 122.

Referring particularly to FIG. 2 and FIG. 3, the outer layer 112 of the guidewire 100 comprises and is defined by an outer surface 114 of the outer layer 112, and further comprises an opposing inner surface 116 of the outer layer 112. The outer layer 112 further comprises one or more than one circumferential wire 124 comprising a material that generates a magnetic field in response to the application of electric current. In a preferred embodiment, the one or more than one circumferential wire 124 is a plurality of circumferential wires 124. As can be seen, particularly in FIG. 2, the one or more than one circumferential wire 124 is oriented in a circumferential spiral around the central fluid layer 118. In one embodiment, the one or more than one circumferential wire 124 comprises copper or iron. In a preferred embodiment, the one or more than one circumferential wire 124 comprises insulated copper or insulated iron. In one embodiment, the outer layer 112 further comprises one or more than one accessory wire 126 encircling each of the one or more than one circumferential wire 124. In one embodiment, the one or more than one accessory wire 126 comprises copper. In a preferred embodiment, the one or more than one circumferential wire 124 comprises iron and the one or more than one accessory wire 126 comprises copper. In a preferred embodiment, not shown, the outer layer 112 further comprises a plurality of longitudinally directed ribs 128 comprising a material that generates a magnetic field in response to the application of electric current. The ribs 128 permit the generation of a magnetic field in addition to the magnetic field generated by the one or more than one circumferential wire 124. In one embodiment, the ribs 128 comprise copper or iron. In a preferred embodiment, the ribs 128 comprise insulated copper or insulated iron. In one embodiment, the outer layer 112 further comprises between two ribs 128 and ten ribs 128. In another embodiment, the outer layer 112 further comprises between two ribs 128 and six ribs 128. In a preferred embodiment, the outer layer 112 comprises four ribs 128.

In one embodiment, the outer layer 112 further comprises a biocompatible material, such as for example an epoxy resin or a polyurethane resin, that allows the guidewire 100 to bend or flex sufficiently for the intended purposes, and that allows use of the guidewire 100 during in vivo procedures, such as for example percutaneous transluminal angioplasty, as will be understood by those with skill in the art with reference to this disclosure. Further, the biocompatible material is also selected to allow incorporation and functioning of the one or more than one circumferential wire 124, and when present, the one or more than one accessory wire 126 into the outer layer 112, the one or more than one rib 128 or other components of the guidewire 100, as will be understood by those with skill in the art with reference to this disclosure.

The central fluid layer 118 is defined by the inner surface 116 of the outer layer 112 which forms the outer extent of the central fluid layer 118, and the central fluid layer 118 is surrounded by the outer layer 112. The central fluid layer 118 comprises magnetorheological fluid, that is, a suspension of micrometer-sized magnetic particles 130 suspended in a carrier fluid 132. In a preferred embodiment, the magnetorheological fluid further comprises a surfactant to offset the inherent density difference between the magnetic particles 130 and the carrier fluid 132. The magnetorheological fluid further comprises a viscosity. In one embodiment, the carrier fluid 132 is selected from the group consisting of mineral oil, polyethelene glycol, a synthetic oil and water. In a preferred embodiment, the carrier fluid 132 is silicon oil. In one embodiment, the magnetic particles 130 comprise ferrometallic particles, such as for example carbonyl iron. In one embodiment, the surfactant is selected from the group consisting of citric acid, oleic acid, and soy lecithin.

When present, the proximal seal 120 and the distal seal 122 constrain the magnetorheological fluid within the central fluid layer 118. In a preferred embodiment, the proximal seal 120 and the distal seal 122 comprise the same biocompatible material as the outer layer 112 comprises, such as for example an epoxy resin or a polyurethane resin, and the proximal seal 120 and the distal seal 122 are in continuity with the outer layer 112.

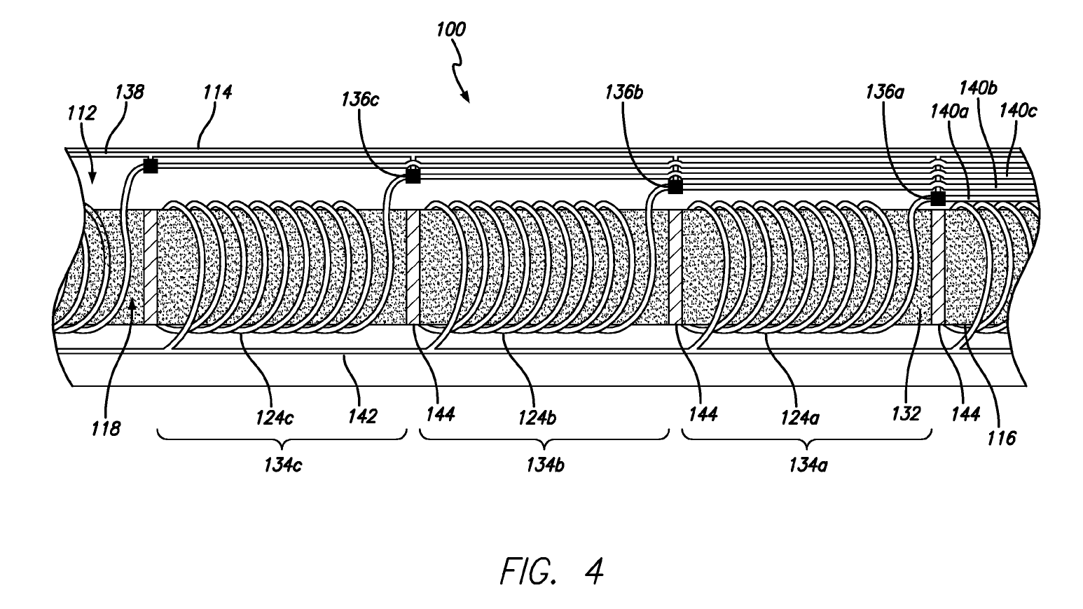

Referring now to FIG. 4, there is shown is a partial, close-up longitudinal cutaway view of another embodiment of the guidewire shown in FIG. 1 taken along the line 4-4. In this embodiment, the guidewire 100 comprises a plurality of segments 134. In one embodiment, the plurality of segments 134 is three segments 134. In one embodiment, the plurality of segments 134 comprises more than three segments 134. In one embodiment, the plurality of segments 134 comprises five or more than five segments 134. In one embodiment, the plurality of segments 134 comprises ten or more than ten segments 134. By way of example, three segments 134 (labeled 134a, 134b and 134c in FIG. 4) of the plurality of segments 134 of the guidewire 100 are shown in FIG. 4. Each segment 134 comprises a separate circumferential wire 124 (labeled 124a, 124b and 124c, respectively, in

FIG. 4) or comprises a plurality of separate circumferential wires 124 that are separate from the circumferential wire or wires in the other segments 134. In one embodiment, the guidewire 100 further comprises one or more than one corresponding accessory wire 126 for each circumferential wire 124 as shown in FIG. 2. In one embodiment, only the proximal section 106 of the guidewire 100 comprises a plurality of segments 134. In one embodiment, only the intermediate section 108 of the guidewire 100 comprises a plurality of segments 134. In one embodiment, only the distal section 110 of the guidewire 100 comprises a plurality of segments 134. In another embodiment, both the proximal section 106 and the intermediate section 108 comprise a plurality of segments 134. In another embodiment, both the intermediate section 108 and the distal section 110 comprise a plurality of segments 134. In another embodiment, both the proximal section 106 and the distal section 110 comprise a plurality of segments 134. In another embodiment, all three sections, the proximal section 106, the intermediate section 108 and the distal section 110 comprise a plurality of segments 134. Each segment of the plurality of segments 134 further comprises an electronic gate 136 (labeled 136a, 136b and 136c, respectively, in FIG. 4). In one embodiment, the electronic gate 136 is a field effect transistor (FET) located between adjoining segments 134 of the plurality of segments. Each segment 134 further comprises a source wire 138 connected to each electronic gate 136, one or more than one gating wire 140 (labeled 140a, 140b and 140c, respectively, in FIG. 4) connected to each electronic gate 136, and a drain wire 142 connected to all of the circumferential wires 124. In one embodiment, each segment 134 of the plurality of segments is separated from an adjoining segment 134 by a barrier 144, where each barrier comprises a material that prevents the magnetorheological fluid in one segment 134 from communicating with the magnetorheological fluid in the adjoining segment 134. In one embodiment, the barrier 144 comprises polyurethane. As will be understood by those with skill in the art with reference to this disclosure, an electric signal from the gating wire 140 allows electric current to flow from the source wire 138 to the drain wire 142 through each electronic gate 136 and the circumferential wire 124. Further as will be understood by those with skill in the art with reference to this disclosure, the current in each circumferential wire 124 can be varied independently from the current in the other circumferential wires 124. The viscosity of the magnetorheological fluid in each segment 134 is dependent on the current in each circumferential wire 124, and the stiffness of each segment 134 is dependent on the viscosity of the magnetorheological fluid in each segment 134. Therefore, the stiffness in each segment 134 can be varied independently from the stiffness in other segments 134 by varying the current in each circumferential wire 124.

According to another embodiment of the present invention, there is provided a system for performing a procedure within a human body, such as for example percutaneous transluminal angioplasty. Referring again to FIG. 1, the system 146 comprises one or more than one guidewire 100 according to the present invention. The system 146 further comprises a control 148 for controllably sending electric current through the one or more than one circumferential wire 124, and for controllably sending electric current through the one or more than one accessory wire 126 when one or more than one accessory wire 126 is present. The control 148 is connected to the guidewire 100 through the proximal section 106 of the guidewire 100 by one or more than one conduit 150, such as for example one or more than one connector wire as shown in FIG. 1.

In one embodiment, there is provided a method for performing a procedure within a human body, such as for example percutaneous transluminal angioplasty. In one embodiment, the method comprises providing a guidewire according to the present invention and using the guidewire to perform the procedure. In one embodiment, the method comprises providing a system according to the present invention, generating electric current through the control and sending electric current through the one or more than one circumferential wire and, when present, the one or more than one accessory wire. The electric current in the one or more than one circumferential wire and, when present, in the one or more than one accessory wire or the one or more than one rib or both the one or more than one accessory wire and the one or more than one rib, creates a magnetic flux in the fluid layer causing the magnetic particles suspended in the carrier fluid to become aligned and, thereby increases the fluid viscosity by decreasing the ability of the carrier fluid to flow around the particles. This controllably increases the stiffness of the guidewire.

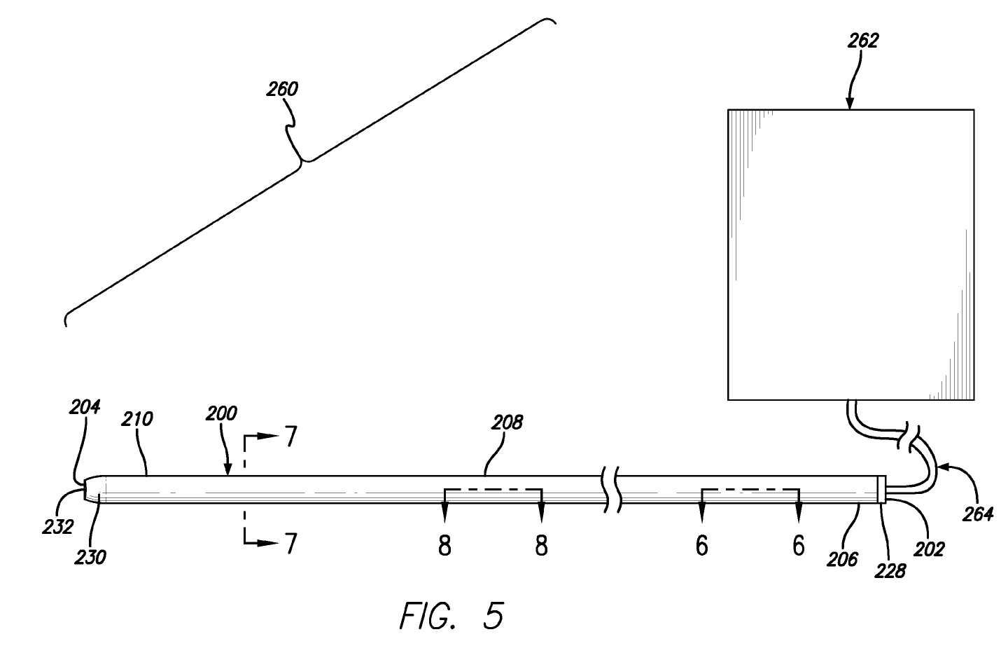

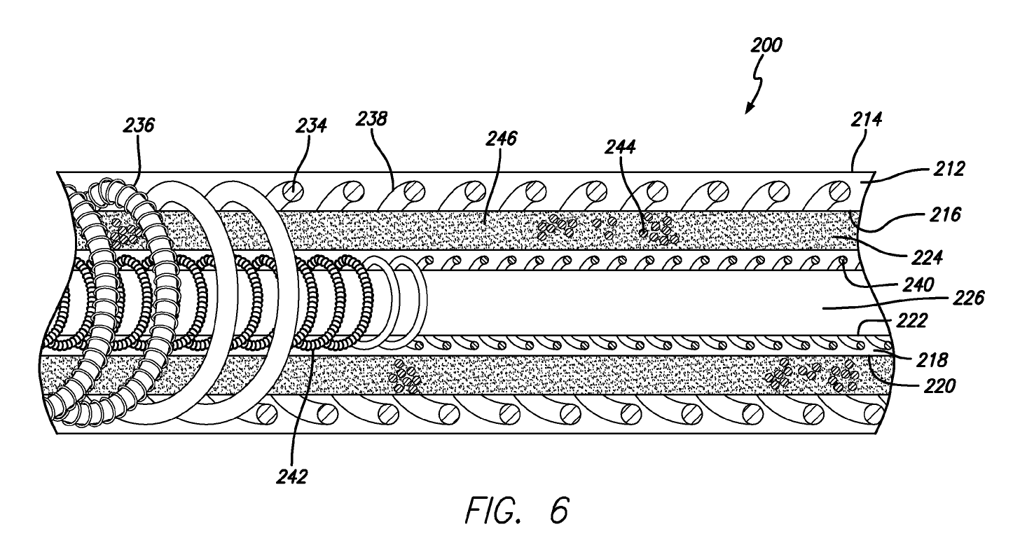

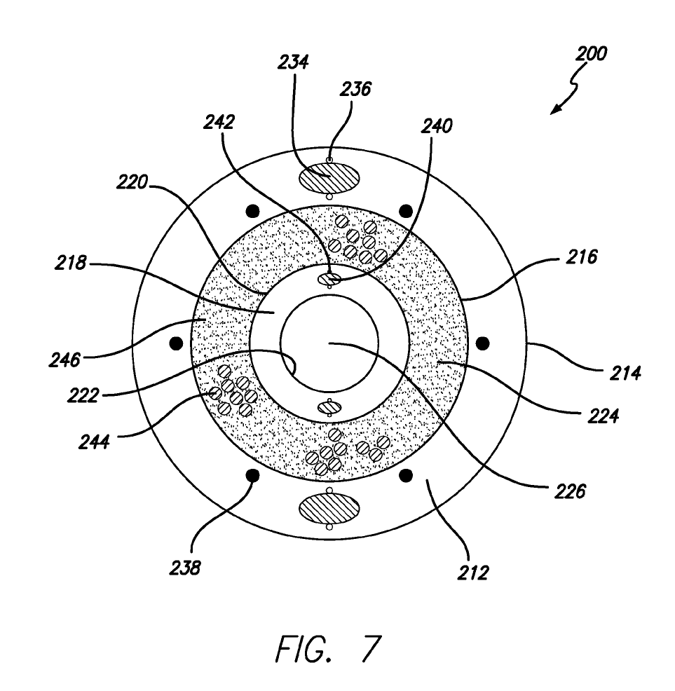

According to another embodiment of the present invention, there is provided a catheter with a stiffness that can be varied during use inside a human body. The catheter comprises a magnetorheological fluid. Referring now to FIG. 5, FIG. 6 and FIG. 7, there are shown, respectively, a partial, lateral perspective view of one embodiment of a system according to the present invention for performing a procedure within a human body, where the system comprises a catheter according to the present invention (FIG. 5); a partial, close-up longitudinal cutaway view of one embodiment of the catheter shown in FIG. 5 taken along the line 6-6 (FIG. 6); and a cross-sectional view of one embodiment of the catheter shown in FIG. 5 taken along the line 7-7 (FIG. 7). As can be seen, the catheter 200 comprises a proximal end 202 and a distal end 204, and further comprises, oriented from the proximal end 202 to the distal end 204, a proximal section 206, an intermediate section 208 and a distal section 210, where the intermediate section 208 connects the proximal section 206 to the distal section 210. The catheter 200 further comprises an outer layer 212 comprising and defined by an outer surface 214 of the outer layer 212, and an opposing inner surface 216 of the outer layer 212. The catheter 200 further comprises an inner layer 218 comprising and defined by an outer surface 220 of the inner layer 218, and an opposing inner surface 222 of the inner layer 218. The catheter 200 further comprises a fluid layer 224 between and defined by the inner surface 216 of the outer layer 212, and the outer surface 220 of the inner layer 218. The catheter 200 further comprises a central lumen 226 defined by the inner surface 222 of the inner layer 218. The catheter 200 further comprises a longitudinal length between the proximal end 202 and the distal end 204. In one embodiment, the longitudinal length is between 50 cm and 250 cm. In another embodiment, the longitudinal length is between 100 cm and 200 cm. The catheter 200 further comprises a cross-sectional area defined by the outer surface 214 of the outer layer 212. In one embodiment, the cross-sectional area of the catheter 200 is between 0.2 mm² and 7 mm² In another embodiment, the cross-sectional area of the catheter 200 is between 0.2 mm² and 3.5 mm²

The proximal section 206 of the catheter 200 comprises a proximal seal 228 for sealing the fluid layer 224 at the proximal end 202 of the catheter 200. In one embodiment, the proximal seal 228 is configured to connect to a surgical instrument once the surgical instrument is inserted into the central lumen 226 of the catheter 200, as will be understood by those with skill in the art with reference to this disclosure.

In one embodiment, the distal section 210 of the catheter 200 comprises a distal seal 230 for sealing the fluid layer 224 at the distal end 204 of the catheter 200. In a preferred embodiment, the distal section 210 is tapered as shown in FIG. 5. In another preferred embodiment, the distal section 210 has an opening 232 in continuity of the central lumen 226 to permit passage of a guidewire or a surgical instrument within the central lumen 226 externally, as will be understood by those with skill in the art with reference to this disclosure.

Referring particularly to FIG. 6 and FIG. 7, the outer layer 212 of the catheter 200 comprises and is defined by an outer surface 214 of the outer layer 212, and further comprises an opposing inner surface 216 of the outer layer 212. The outer layer 212 further comprises one or more than one circumferential wire 234 comprising a material that generates a magnetic field in response to the application of electric current. In a preferred embodiment, the one or more than one circumferential wire 234 is a plurality of circumferential wires 234. As can be seen, particularly in FIG. 6, the one or more than one circumferential wire 234 is oriented in a circumferential spiral around the fluid layer 224. In one embodiment, the one or more than one circumferential wire 234 comprises copper or iron. In a preferred embodiment, the one or more than one circumferential wire 234 comprises insulated copper or insulated iron. In one embodiment, as shown in FIG. 6, the outer layer 212 further comprises one or more than one accessory wire 236 encircling each of the one or more than one circumferential wire 234. In one embodiment, the one or more than one accessory wire 236 comprises copper. In a preferred embodiment, the one or more than one circumferential wire 234 comprises iron and the one or more than one accessory wire 236 comprises copper. In a preferred embodiment, as shown in FIG. 7, the outer layer 212 further comprises a plurality of longitudinally directed ribs 238 comprising a material that generates a magnetic field in response to the application of electric current. The ribs 238 permit the generation of a magnetic field in addition to the magnetic field generated by the one or more than one circumferential wire 234. In one embodiment, the ribs 238 comprise copper or iron. In a preferred embodiment, the ribs 238 comprise insulated copper or insulated iron. In one embodiment, the outer layer 212 further comprises between four ribs 238 and twenty ribs 238. In another embodiment, the outer layer 212 further comprises between six ribs 238 and fifteen ribs 238. In another embodiment, the outer layer 212 comprises twelve ribs 238.

The outer layer 212 comprises a biocompatible material, such as for example an epoxy resin or a polyurethane resin, that allows the catheter 200 to bend or flex sufficiently for the intended purposes, and that allows use of the catheter 200 during in vivo procedures, such as for example percutaneous transluminal angioplasty, as will be understood by those with skill in the art with reference to this disclosure. Further, the biocompatible material is also selected to allow incorporation of the one or more than one circumferential wire 234 and, when present, the one or more than one accessory wire 236 and the ribs 238 into the outer layer 212 or other components of the catheter 200, as will be understood by those with skill in the art with reference to this disclosure.

The inner layer 218 comprises and is defined by an outer surface 220 of the inner layer 218, and an opposing inner surface 222 of the inner layer 218. The inner layer 218 comprises a biocompatible material, such as for example an epoxy resin or a polyurethane resin, that allows the catheter 200 to bend or flex sufficiently for the intended purposes, and that allows use of the catheter 200 during in vivo procedures, such as for example percutaneous transluminal angioplasty, as will be understood by those with skill in the art with reference to this disclosure. In one embodiment, the inner surface 222 of the inner layer 218 further comprises a lubricious coating adjoining the central lumen 226, such as for example polytetrafluoroethylene, to decrease frictional resistance to guidewires or other devices passing through the central lumen 226. In one embodiment, the inner layer 218 further comprises one or more than one circumferential wire 240 comprising a material that generates a magnetic field in response to the application of electric current. In one embodiment, the one or more than one circumferential wire 240 is a plurality of wires 240. As can be seen, particularly in FIG. 6, the one or more than one circumferential wire 240, when present, is oriented in a circumferential spiral between the fluid layer 224 and the central lumen 226. In one embodiment, the one or more than one circumferential wire 240 comprises copper or iron. In a preferred embodiment, the one or more than one circumferential wire 240 comprises insulated copper or insulated iron. In one embodiment, the inner layer 218 further comprises one or more than one accessory wire 242 encircling each of the one or more than one circumferential wire 240. In one embodiment, the one or more than one accessory wire 242 comprises copper. When the inner layer 218 comprises one or more than one circumferential wire 240, or comprises one or more than one circumferential wire 240 encircled by one or more than one accessory wire 242, the inner layer 218 must comprise a material that allows incorporation of the one or more than one circumferential wire 240, and the one or more than one accessory wire 242 into the inner layer 218, as will be understood by those with skill in the art with reference to this disclosure.

The fluid layer 224 is between and defined by the inner surface 216 of the outer layer 212, and the outer surface 220 of the inner layer 218. The fluid layer 224 comprises magnetorheological fluid, that is, a suspension of micrometer-sized magnetic particles 244 suspended in a carrier fluid 246. In a preferred embodiment, the magnetorheological fluid further comprises a surfactant to offset the inherent density difference between the magnetic particles 244 and the carrier fluid 246. The magnetorheological fluid comprises a viscosity. The magnetorheological fluid maintains the transverse separation of the inner surface 216 of the outer layer 212, and the outer surface 220 of the inner layer 218, thereby maintaining the fluid layer 224 at a constant transverse thickness. In one embodiment, the magnetorheological fluid is selected from the group consisting of mineral oil, polyethelene glycol, a synthetic oil and water. In a preferred embodiment, the magnetorheological fluid is silicon oil. In one embodiment, the magnetic particles 244 comprise ferrometallic particles, such as for example carbonyl iron. In one embodiment, the surfactant is selected from the group consisting of citric acid, oleic acid, and soy lecithin.

The central lumen 226 comprises a cross-sectional area defined by the inner surface 222 of the inner layer 218. In one embodiment, the cross-sectional area of the central lumen 226 is between 1.2 mm² and 4.2 mm² In another embodiment, the cross-sectional area of the central lumen 226 is between 1.7 mm² and 3.2 mm²

When present, the proximal seal 228 and the distal seal 230 constrain the magnetorheological fluid within the fluid layer 224. In a preferred embodiment, the proximal seal 228 and the distal seal 230 comprise the same biocompatible material as the outer layer 212 comprises, such as for example an epoxy resin or a polyurethane resin, and the proximal seal 228 and the distal seal 230 are in continuity with the outer layer 212.

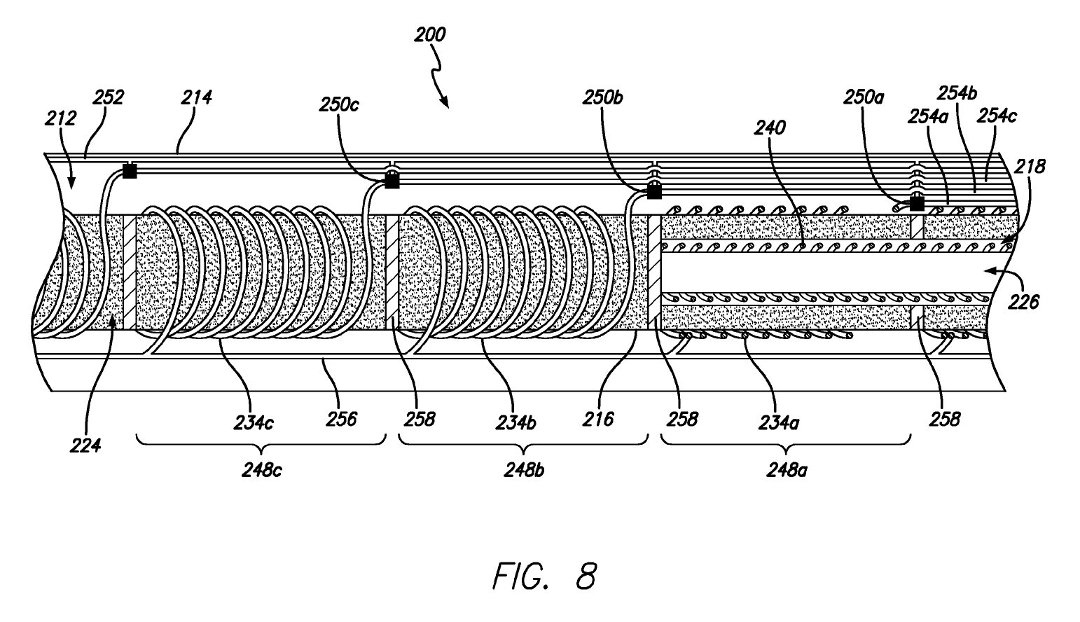

Referring now to FIG. 8, there is shown is a partial, close-up longitudinal cutaway view of another embodiment of the catheter shown in FIG. 5 taken along the line 8-8. In this embodiment, the catheter 200 comprises a plurality of segments 248. In one embodiment, the plurality of segments 248 comprises three segments 248. In one embodiment, the plurality of segments 248 comprises more than three segments 248. In one embodiment, the plurality of segments 248 comprises five or more than five segments 248. In one embodiment, the plurality of segments 248 comprises ten or more than ten segments 248. By way of example, three segments 248 (labeled 248a, 248b and 248c in FIG. 8) of the plurality of segments 248 of the catheter 200 are shown in FIG. 8. Each segment 248 comprises a separate circumferential wire 234 (labeled 234a, 234b and 234c, respectively, in FIG. 8) in the outer layer 212 or comprises a plurality of circumferential wires 234 in the outer layer 212 that are separate from the other segments 248. In one embodiment, the catheter 200 further comprises a corresponding accessory wire 236 or corresponding accessory wires 236 for each circumferential wire 234. In one embodiment, only the proximal section 206 of the catheter 200 comprises a plurality of segments 248. In one embodiment, only the intermediate section 208 of the catheter 200 comprises a plurality of segments 248. In one embodiment, only the distal section 210 of the catheter 200 comprises a plurality of segments 248. In another embodiment, both the proximal section 206 and the intermediate section 208 comprise a plurality of segments 248. In another embodiment, both the intermediate section 208 and the distal section 210 comprise a plurality of segments 248. In another embodiment, both the proximal section 206 and the distal section 210 comprise a plurality of segments 248. In another embodiment, all three sections, the proximal section 206, the intermediate section 208 and the distal section 210 comprise a plurality of segments 248. Each segment of the plurality of segments further comprises an electronic gate 250 (labeled 250a, 250b and 250c, respectively, in FIG. 8). In one embodiment, the electronic gate 250 is a field effect transistor (FET) located between adjoining segments 248. The plurality of segments 248 further comprise a source wire 252 connected to each electronic gate 250, one or more than one gating wire 254 (labeled 254a, 254b and 254c, respectively, in FIG. 8) connected to each electronic gate 250, and a drain wire 256 connected to all of the circumferential wires 234. In one embodiment, each segment 248 is separated from an adjoining segment 248 by a barrier 258, where each barrier comprises a material that prevents the magnetorheological fluid in one segment 248 from communicating with the magnetorheological fluid in the adjoining segment 248. In one embodiment, the barrier 258 comprises polyurethane. As will be understood by those with skill in the art with reference to this disclosure, an electric signal from the gating wire 254 allows electric current to flow from the source wire 252 to the drain wire 256 through each electronic gate 250 and the circumferential wire 234. Further as will be understood by those with skill in the art with reference to this disclosure, the current in each circumferential wire 234 can be varied independently from the current in the other circumferential wires 234. The viscosity of the magnetorheological fluid in each segment 248 is dependent on the current in each circumferential wire 234, and the stiffness of each segment 248 is dependent on the viscosity of the magnetorheological fluid in each segment 248. Therefore, the stiffness in each segment 248 can be varied independently from the stiffness in other segments 248.

According to another embodiment of the present invention, there is provided a system for performing a procedure within a human body, such as for example percutaneous transluminal angioplasty. Referring again to FIG. 5, the system 260 comprises one or more than one catheter 200 according to the present invention. The system 260 further comprises a control 262 for controllably sending electric current through the one or more than one circumferential wire 234, and for controllably sending electric current through the one or more than one accessory wire 236, circumferential wire 240, accessory wire 242 and the ribs 238 when present. The control 262 is connected to the catheter 200 through the proximal section 206 of the catheter 200 by one or more than one conduit 264, such as for example one or more than one connector wire as shown in FIG. 5.

In one embodiment, there is provided a method for performing a procedure within a human body, such as for example percutaneous transluminal angioplasty. In one embodiment, the method comprises providing a catheter according to the present invention and using the catheter to perform the procedure. In one embodiment, the method comprises providing a system according to the present invention, generating electric current through the control and sending electric current through the one or more than one circumferential wire and, when present, the one or more than one accessory wire and the ribs. The electric current in the one or more than one circumferential wire and, when present, in the one or more than one accessory wire and the ribs, creates a magnetic flux in the fluid layer causing the magnetic particles suspended in the carrier fluid to become aligned and thereby increases the fluid viscosity by decreasing the ability of the carrier fluid to flow around the particles. This controllably increases the stiffness of the catheter.

As will be understood by those with skill in the art with reference to this disclosure, the viscosity of the magnetorheological fluid can be changed very accurately by applying a magnetic field to the magnetorheological fluid and by varying the intensity of the magnetic field, thereby varying the stiffness of the guidewire or of the catheter according to the present invention.

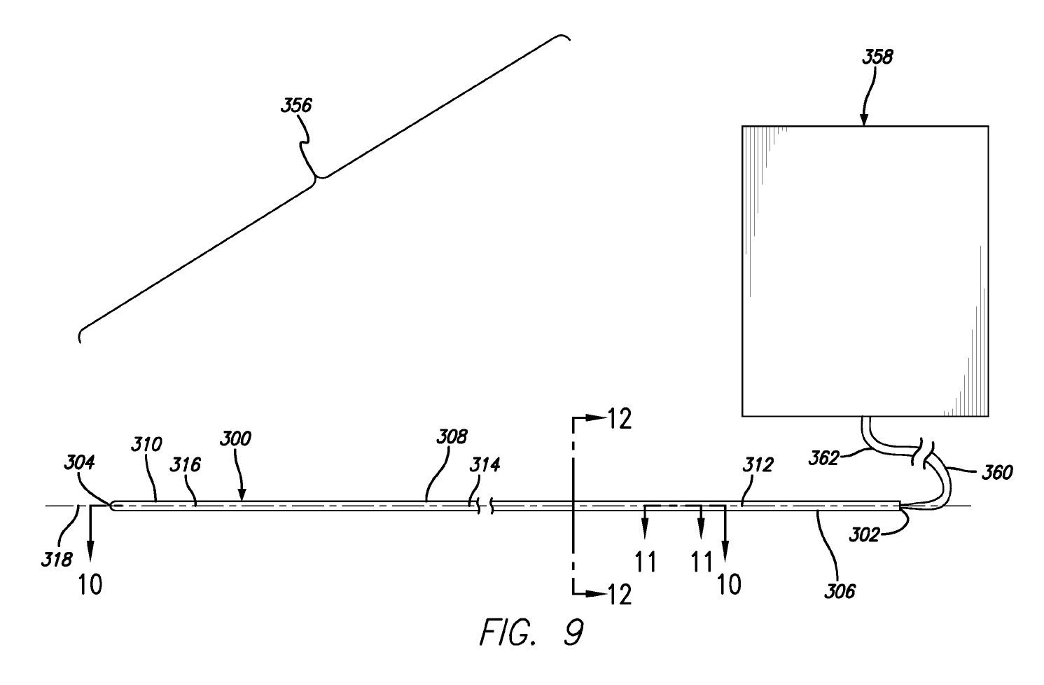

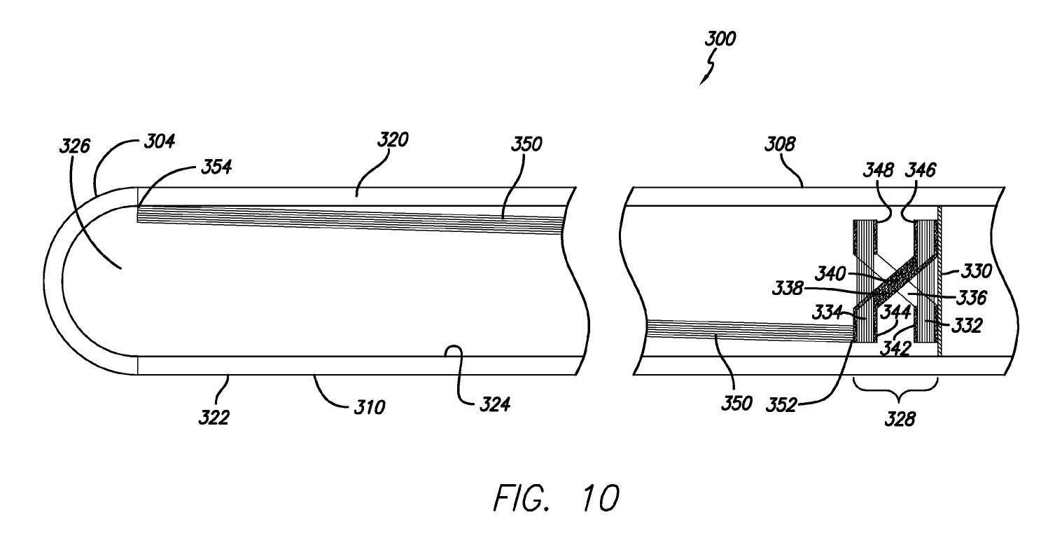

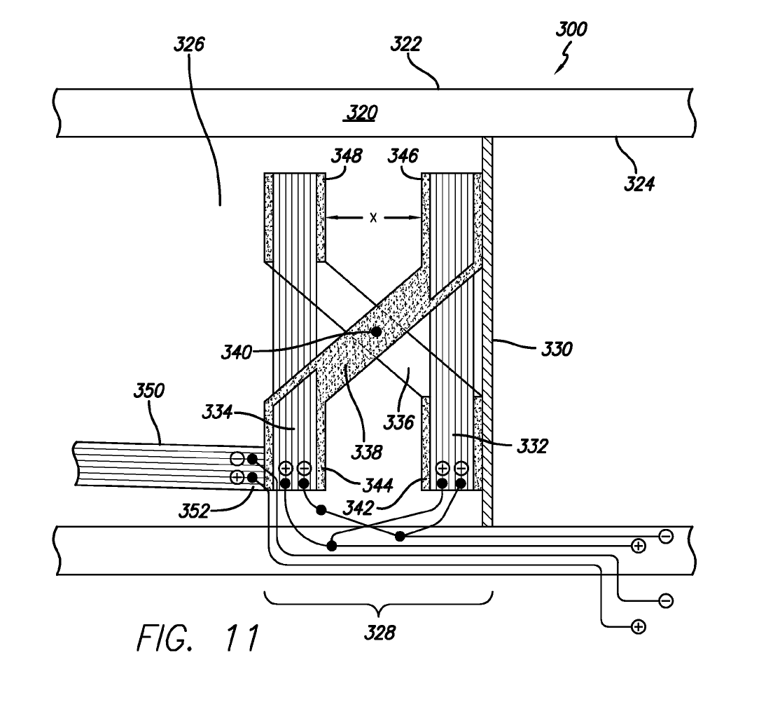

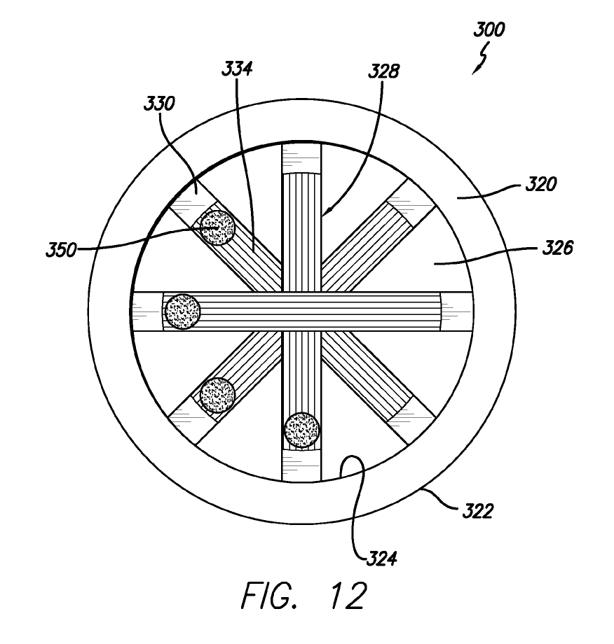

According to one embodiment of the present invention, there is provided a guidewire that can better negotiate the curves of the arterial lumen during an endovascular procedure than standard guidewires, by being able to controllably bend during use. Referring now to FIG. 9, FIG. 10, FIG. 11 and FIG. 12, there are shown, respectively, a partial, lateral perspective view of one embodiment of a system according to the present invention for performing a procedure within a human body, where the system comprises a guidewire according to the present invention comprising a plurality of piezoelectric elements (FIG. 9); a partial, close-up longitudinal cutaway view of one embodiment of the guidewire shown in FIG. 9 taken along the line 10-10 (FIG. 10); another partial, close-up longitudinal cutaway view of one embodiment of the guidewire shown in FIG. 9 and FIG. 10 taken along the line 11-11 (FIG. 11); and a cross-sectional view of one embodiment of a guidewire according to the present invention, taken along the line 12-12 (FIG. 12). As can be seen, the guidewire 300 comprises a proximal end 302 and a distal end 304, and further comprises, oriented from the proximal end 302 to the distal end 304, a proximal section 306, an intermediate section 308 and a distal section 310, where the intermediate section 308 connects the proximal section 306 to the distal section 310. The guidewire 300 further comprises a longitudinal axis 312 of the proximal section 306, a longitudinal axis 314 of the intermediate section 308, and a longitudinal axis 316 of the distal section 310, where the longitudinal axis 314 of the intermediate section 308 connects the longitudinal axis 312 of the proximal section 306 to the longitudinal axis 316 of the distal section 310, and where the longitudinal axis of the proximal section 312, the longitudinal axis of the intermediate section 314, and the longitudinal axis of the distal section 316 define a longitudinal axis 318 of the guidewire 300. The guidewire 300 further comprises a longitudinal length between the proximal end 302 and the distal end 304. In one embodiment, the longitudinal length of the guidewire 300 is between 50 cm and 250 cm. In another embodiment, the longitudinal length of the guidewire 300 is between 100 cm and 200 cm.

The guidewire 300 further comprises one or more than one outer layer 320 comprising and defined by an outer surface 322 of the outer layer 320, and an opposing inner surface 324 of the outer layer 320. The guidewire 300 further comprises a central layer 326 defined by the inner surface 324 of the outer layer 320, and surrounded by the outer layer 320. The guidewire 300 further comprises a cross-sectional area defined by the outer surface 322 of the outer layer 320. In one embodiment, the cross-sectional area is between 0.4 mm² and 4.0 mm² In another embodiment, the cross-sectional diameter is between 0.4 mm² and 1.0 mm² In a preferred embodiment, the distal section 310 of the guidewire 300 is tapered at the distal end 304 of the guidewire 300 as shown in FIG. 9.

The outer layer 320 comprises a biocompatible material, such as for example an epoxy resin or a polyurethane resin, that allows the guidewire 300 to bend or flex sufficiently for the intended purposes, and that allows use of the guidewire 300 during in vivo procedures, such as for example percutaneous transluminal angioplasty, as will be understood by those with skill in the art with reference to this disclosure.

The guidewire 300 further comprises one or more than one actuator base 328. Referring again to FIG. 10, FIG. 11 and FIG. 12, each actuator base 328 comprises a base plate 330, a first piezoelectric core 332, a second piezoelectric core 334, a first brace 336, a second brace 338, and a pivot 340 connecting the first brace 336 to the second brace 338. The base plate 330 spans the central layer 326 between the inner surface 324 of the outer layer 320 and is attached to two opposing locations of the inner surface 324 of the outer layer 320. In one embodiment, the base plate 330 comprises polyurethane or an epoxy resin composite.

The first piezoelectric core 332 is adjacent the base plate 330, and extends across the central layer 326 between the inner surface 324 of the outer layer 320 without contacting the inner surface 324 of the outer layer 320 at any location. The second piezoelectric core 334 extends across the central layer 326 between the inner surface 324 of the outer layer 320 without contacting the inner surface 324 of the outer layer 320 at any location. The first piezoelectric core 332 comprises a long axis, and the second piezoelectric core 334 comprises a long axis. The second piezoelectric core 334 is spaced apart from the first piezoelectric core 332 at a distance x, and the long axis of the first piezoelectric core 332 is parallel to the long axis of the second piezoelectric core 334 as can be seen in FIG. 10 and FIG. 11. The first piezoelectric core 332 and the second piezoelectric core 334 comprise a piezoelectric material.

The first brace 336 comprises a proximal end and a distal end. The second brace 338 comprises a proximal end and a distal end. The first brace 336 comprises a first casing 342 at the proximal end of the first brace 336 and a second casing 344 at the distal end of the first brace 336. The second brace 338 comprises a first casing 346 at the proximal end of the second brace 338 and a second casing 348 at the distal end of the second brace 338. The first casing 342 of the first brace 336 and the first casing 346 of the second brace 338 surround opposing ends of the first piezoelectric core 332. The second casing 344 of the first brace 336 and the second casing 348 of the second brace 338 surround opposing ends of the second piezoelectric core 334.

The first casing 342 of the first brace 336 comprises a first portion of a first connector and the base plate 330 comprises a second portion of a first connector. The first casing 346 of the second brace 338 comprises a first portion of a second connector and the base plate 330 comprises a second portion of a second connector. The first connector and the second connector allow the first piezoelectric core 332 to expand longitudinally (increasing longitudinal dimension toward the inner surface 324 of the outer layer 320) while maintaining a relative distance to the base plate 330. In one embodiment, the first portion of the first connector and the first portion of the second connector are male type extensions, and the second portion of the first connector and the second portion of the second connector are female type portions such as slotted grooves which mate with the male type extensions. In one embodiment, the guidewire 300 further comprises micrometer sized ball bearings between the first portion of the first connector and the second portion of the first connector, or between the first portion of the second connector and the second portion of the second connector to decrease friction. In another embodiment, the first portion of the first connector and the second portion of the first connector, or the first portion of the second connector and the second portion of the second connector comprise a polytetrafluoroethylene (PTFE) or similar coating to decrease friction.

The first brace 336 and the second brace 338 comprise a material suitable for the intended use of the guidewire 300, as will be understood by those with skill in the art with reference to this disclosure. In one embodiment, the first brace 336 and the second brace 338 comprise metal such as iron or aluminum, or comprise a plastic such as hardened polyurethane. In one embodiment, the first casing 342 of the first brace 336, the second casing 344 of the first brace 336, the first casing 346 of the second brace 338 and the second casing 348 of the second brace 338 each comprise a plastic such as hardened polyurethane.

The pivot 340 of the actuator base 328 joins the center of the first brace 336 to the center of the second brace 338 as can be seen in FIG. 11. In one embodiment, the pivot 340 is a screw comprising a head at one end and a distal end extending through matching holes in the center of the first brace 336 and the center of the second brace 338, where the pivot comprises a transverse hole in the distal end to receive a pin, thereby fixing the pivot 340 in place with respect to the first brace 336 and the second brace 338. In one embodiment, the pivot 340 comprises metal such as iron or aluminum, or comprises a plastic such as hardened polyurethane.

The guidewire 300 further comprises one piezoelectric strut 350 associated with each actuator base 328. The piezoelectric strut 350 comprises a proximal end 352 and a distal end 354. The proximal end 352 of the piezoelectric strut 350 is connected to either the second casing 344 of the first brace 336 or, as shown in FIG. 10 and FIG. 11, the second casing 348 of the second brace 338. The distal end 354 of the piezoelectric strut 350 is connected to the inner surface 324 of the outer layer 320 in the distal section 310 of the guidewire 300. The piezoelectric strut 350 comprises a piezoelectric material.

In one embodiment, the guidewire 300 comprises one actuator base 328 and associated piezoelectric strut 350. In one embodiment, the guidewire 300 comprises a plurality of actuator bases 328 and associated piezoelectric struts 350. In one embodiment, the guidewire 300 comprises two actuator bases 328 and associated piezoelectric struts 350. In one embodiment, the guidewire 300 comprises three actuator bases 328 and associated piezoelectric struts 350. In one embodiment, the guidewire 300 comprises four actuator bases 328 and associated piezoelectric struts 350. In one embodiment, the guidewire 300 comprises more than four actuator bases 328 and associated piezoelectric struts 350.

According to another embodiment of the present invention, there is provided a system for performing a procedure within a human body, such as for example percutaneous transluminal angioplasty. Referring again to FIG. 9, the system 356 comprises one or more than one guidewire 300 according to the present invention, and one or more than one control 358 for controlling the function of the guidewire 300.

The guidewire 300 further comprises wiring 360 connecting the first piezoelectric core 332 and the second piezoelectric core 334 to the control 358 for controlling transmission of current from the control 358 to the first piezoelectric core 332 and the second piezoelectric core 334. The guidewire 300 further comprises wiring 362 connecting the piezoelectric strut 350 to the control 358 for controlling transmission of current from the control to the piezoelectric strut 350. In a preferred embodiment, both the wiring 360 and the wiring 362 traverse at least part of the outer layer 320 of the guidewire 300 as shown in FIG. 11. In one embodiment, the guidewire 300 comprises separate wiring 360 connecting the first piezoelectric core 332 and the second piezoelectric core 334 of each actuator base 328 to the control 358 for controlling transmission of current from the control 358 to the first piezoelectric core 332 and the second piezoelectric core 334. In one embodiment, the guidewire 300 comprises separate wiring 362 connecting the piezoelectric strut 350 associated with each actuator base 328 to the control 358 for controlling transmission of current from the control 358 to the piezoelectric strut 350.

Referring again to FIG. 12, there is shown a cross-sectional view of one embodiment of the guidewire according to the present invention. In this embodiment, the guidewire 300 comprises a plurality of actuator bases 328, each actuator base 328 associated with one piezoelectric strut 350. As can be seen, the actuator bases 328 are spaced apart from each other longitudinally, and are rotated relative to one another axially, thereby causing the distal end 354 of each piezoelectric strut 350 to be connected to the inner surface 324 of the outer layer 320 in the distal section 310 of the guidewire 300 at a different location. As can be seen, in a preferred embodiment, the transverse widths of the individual elements of each actuator base 328 are kept to a minimum so as not to interfere with the axial passage of the piezoelectric strut 350 associated with another actuator base 328.

According to another embodiment of the present invention, there is provided a method for performing an endovascular procedure in a patient. The method comprises, first providing a guidewire, such as a guidewire 300 according to the present invention. Next, the guidewire 300 in a first configuration is inserted into an arterial lumen of the patient percutaneously, according to techniques as will be understood by those with skill in the art with reference to this disclosure. When inserted, current is being supplied through wiring 362 to the one or more than one piezoelectric strut 350 rendering the one or more than one piezoelectric strut 350 in a first longitudinal length, and no current is being supplied through wiring 360 to the one or more than one first piezoelectric core 332 and the second piezoelectric core 334 rendering them in a first longitudinal length, where the longitudinal axis 316 of the distal section 310 of the guidewire 300 is coincident with the longitudinal axis 314 of the intermediate section 308 of the guidewire 300. Then, in order to negotiate a curve of the arterial lumen, the guidewire 300 is changed to a second configuration by supplying current to the wiring 360 to the one or more than one first piezoelectric core 332 and the second piezoelectric core 334 rendering them in a second longitudinal length and increasing the distance x, the axial separation of first piezoelectric core 332 and the second piezoelectric core 334 and decreasing the separation of the first casing 342 of the first brace 336 from the first casing 346 of the second brace 338, and the separation of the second casing 344 of the first brace 336 from the second casing 348 of the second brace 338, and by simultaneously ceasing to supply current to the wiring 362 to the one or more than one piezoelectric strut 350 rendering the one or more than one piezoelectric strut 350 in a second longitudinal length, where the first longitudinal length of the one or more than one first piezoelectric core 332 and the second piezoelectric core 334 is longer than the second longitudinal length of the one or more than one first piezoelectric core 332 and the second piezoelectric core 334, and where the first longitudinal length of the one or more than one piezoelectric strut 350 is shorter than the second longitudinal length of the one or more than piezoelectric strut 350, thereby causing the longitudinal axis 316 of the distal section 310 of the guidewire 300 to become non-coincident with the longitudinal axis 314 of the intermediate section 308 of the guidewire 300. In one embodiment, the current supplied is DC current.

In one embodiment, the first longitudinal length of the one or more than one first piezoelectric core 332 and the second piezoelectric core 334 is longer than the second longitudinal length of the one or more than one first piezoelectric core 332 and the second piezoelectric core 334 by at least 25%. In another embodiment, the first longitudinal length of the one or more than one first piezoelectric core 332 and the second piezoelectric core 334 is longer than the second longitudinal length of the one or more than one first piezoelectric core 332 and the second piezoelectric core 334 by at least 50%. In another embodiment, the first longitudinal length of the one or more than one first piezoelectric core 332 and the second piezoelectric core 334 is longer than the second longitudinal length of the one or more than one first piezoelectric core 332 and the second piezoelectric core 334 by at least 100%. In one embodiment, the second longitudinal length of the one or more than one piezoelectric strut 350 is longer than the first longitudinal length of the one or more than piezoelectric strut 350 by at least 25%. In one embodiment, the second longitudinal length of the one or more than one piezoelectric strut 350 is longer than the first longitudinal length of the one or more than piezoelectric strut 350 by at least 50%. In one embodiment, the second longitudinal length of the one or more than one piezoelectric strut 350 is longer than the first longitudinal length of the one or more than piezoelectric strut 350 by at least 100%.

According to another embodiment of the present invention, there is provided a guidewire with a stiffness that can be varied inside a human body, and comprising a proximal section, a distal section, and an intermediate section between the proximal section and the distal section, where the intermediate section comprises a longitudinal axis, and the distal section comprises a longitudinal axis, and where the longitudinal axis of the distal section can be coincident with the longitudinal axis of the intermediate section or can be controllably made non-coincident with the longitudinal axis of the intermediate section during use inside a human body. In one embodiment, the guidewire is a combination of the elements disclosed for guidewire 100 and guidewire 300, comprising both a magnetorheological fluid and a plurality of piezoelectric elements.

As will be understood by those with skill in the art with reference to this disclosure, the viscosity of the magnetorheological fluid can be changed very accurately by applying a magnetic field to the magnetorheological fluid and by varying the intensity of the magnetic field, thereby varying the stiffness of the guidewire or of the catheter according to the present invention.

Although the present invention has been discussed in considerable detail with reference to certain preferred embodiments, other embodiments are possible. Therefore, the scope of the appended claims should not be limited to the description of preferred embodiments contained in this disclosure.

1. A guidewire with a stiffness that can be varied during use, the guidewire comprising:
a) a proximal end and a distal end, and oriented from the proximal end to the distal end, a proximal section, an intermediate section and a distal section, where the intermediate section connects the proximal section to the distal section;
b) an outer layer comprising and defined by an outer surface of the outer layer and an opposing inner surface of the outer layer;
c) a central fluid layer defined by the inner surface of the outer layer, and surrounded by the outer layer;
d) a longitudinal length between the proximal end and the distal end; and
e) a cross-sectional area defined by the outer surface of the outer layer;
where the outer layer further comprises one or more than one circumferential wire comprising a material that generates a magnetic field in response to the application of electric current;
where the central fluid layer comprises magnetorheological fluid comprising a suspension of micrometer-sized magnetic particles suspended in a carrier fluid; and
where the central fluid layer further comprises a viscosity.

2-42. (canceled)

43. A system for performing a procedure within a human body, the system comprising:
a) one or more than one guidewire according to claim 1; and
b) a control for controllably sending electric current through the one or more than one circumferential wire;
where the control is connected to the guidewire through the proximal section of the guidewire.

44. A method for performing a procedure within a human body, the method comprising:
a) providing a guidewire according to claim 1; and
b) using the guidewire to perform the procedure.

45. A method for performing a procedure within a human body, the method comprising:
a) providing a system according to claim 43; and
b) generating electric current through the control and sending electric current through the one or more than one circumferential wire, thereby creating a magnetic flux in the fluid layer causing the magnetic particles suspended in the carrier fluid to become aligned which increases the viscosity of the fluid by decreasing the ability of the carrier fluid to flow around the particles, thereby controllably increasing the stiffness of the guidewire.

46. A catheter with a stiffness that can be varied during use inside a human body, the catheter comprising:
a) a proximal end and a distal end, and oriented from the proximal end to the distal end, a proximal section, an intermediate section and a distal section, where the intermediate section connects the proximal section to the distal section;
b) an outer layer comprising and defined by an outer surface of the outer layer, and an opposing inner surface of the outer layer;
c) an inner layer comprising and defined by an outer surface of the inner layer, and an opposing inner surface of the inner layer;
d) a fluid layer between and defined by the inner surface of the outer layer, and the outer surface of the inner layer;
e) a central lumen defined by the inner surface of the inner layer;
f) a longitudinal length between the proximal end and the distal end; and
g) a cross-sectional area defined by the outer surface of the outer layer;
where the outer layer of the catheter comprises and is defined by an outer surface of the outer layer;
where the outer layer further comprises one or more than one circumferential wire comprising a material that generates a magnetic field in response to the application of electric current;
where the fluid layer comprises magnetorheological fluid comprising a suspension of micrometer-sized magnetic particles suspended in a carrier fluid; and
where the magnetorheological fluid comprises a viscosity.

47-102. (canceled)

103. A guidewire than can be controllably bent during use, the guidewire comprising:
a) a proximal end and a distal end, and oriented from the proximal end to the distal end, a proximal section, an intermediate section and a distal section, where the intermediate section connects the proximal section to the distal section;
b) a longitudinal axis of the proximal section, a longitudinal axis of the intermediate section, and a longitudinal axis of the distal section, where the longitudinal axis of the intermediate section connects the longitudinal axis of the proximal section to the longitudinal axis of the distal section, and where the longitudinal axis of the proximal section, the longitudinal axis of the intermediate section, and the longitudinal axis of the distal section define a longitudinal axis of the guidewire;
c) a longitudinal length between the proximal end and the distal end;
d) one or more than one outer layer comprising and defined by an outer surface of the outer layer, and an opposing inner surface of the outer layer;
e) a central layer defined by the inner surface of the outer layer, and surrounded by the outer layer;
f) a cross-sectional area defined by the outer surface of the outer layer;
g) one or more than one actuator base, each actuator base comprising a base plate, a first piezoelectric core, a second piezoelectric core, a first brace, a second brace, and a pivot connecting the first brace to the second brace; and
h) one piezoelectric strut comprising a piezoelectric material associated with each actuator base;
where the base plate spans the central layer between the inner surface of the outer layer and is attached to two opposing locations of the inner surface of the outer layer;
where the first piezoelectric core is adjacent the base plate, and extends across the central layer between the inner surface of the outer layer without contacting the inner surface of the outer layer at any location;
where the second piezoelectric core extends across the central layer between the inner surface of the outer layer without contacting the inner surface of the outer layer at any location;
where the first piezoelectric core comprises a long axis, and the second piezoelectric core comprises a long axis;
where the second piezoelectric core is spaced apart from the first piezoelectric core at a distance x, and the long axis of the first piezoelectric core is parallel to the long axis of the second piezoelectric core;
where the first piezoelectric core and the second piezoelectric core comprise a piezoelectric material;
where the first brace comprises a proximal end and a distal end;
where the second brace comprises a proximal end and a distal end;
where the first brace comprises a first casing at the proximal end of the first brace and a second casing at the distal end of the first brace;
where the second brace comprises a first casing at the proximal end of the second brace and a second casing at the distal end of the second brace;
where the first casing of the first brace and the first casing of the second brace surround opposing ends of the first piezoelectric core;
where the second casing of the first brace and the second casing of the second brace surround opposing ends of the second piezoelectric core;
where the first casing of the first brace comprises a first portion of a first connector and the base plate comprises a second portion of a first connector;
where the first casing of the second brace comprises a first portion of a second connector and the base plate comprises a second portion of a second connector;
where the first connector and the second connector allow the first piezoelectric core to expand longitudinally thereby increasing longitudinal dimension toward the inner surface of the outer layer while maintaining a relative distance to the base plate;
where the piezoelectric strut comprises a proximal end and a distal end;
where the proximal end of the piezoelectric strut is connected to either the second casing of the first brace or the second casing of the second brace; and
where the distal end of the piezoelectric strut is connected to the inner surface of the outer layer in the distal section of the guidewire.

104. A guidewire than can controllably bend during use, the guidewire comprising a plurality of piezoelectric struts comprising a piezoelectric material.

105. The guidewire of claim 103, where the longitudinal length of the guidewire is between 50 cm and 250 cm.

106. The guidewire of claim 103, where the longitudinal length of the guidewire is between 100 cm and 200 cm.

107. The guidewire of claim 103, where the cross-sectional area is between 0.4 mm² and 4.0 mm²

108. The guidewire of claim 103, where the cross-sectional area is between 0.4 mm² and 1.0 mm².

109. The guidewire of claim 103, where the distal section of the guidewire is tapered at the distal end of the guidewire.

110. The guidewire of claim 103, where the outer layer comprises a biocompatible material selected from the group consisting of an epoxy resin and a polyurethane resin.

111. The guidewire of claim 103, where the base plate comprises polyurethane or an epoxy resin composite.

112. The guidewire of claim 103, where the first portion of the first connector and the first portion of the second connector are male type extensions, and the second portion of the first connector and the second portion of the second connector are female type portions such as slotted grooves which mate with the male type extensions.

113. The guidewire of claim 103, further comprising micrometer sized ball bearings between the first portion of the first connector and the second portion of the first connector, or between the first portion of the second connector and the second portion of the second connector to decrease friction.

114. The guidewire of claim 103, where the first portion of the first connector and the second portion of the first connector, or the first portion of the second connector and the second portion of the second connector comprise a coating to decrease friction.

115. The guidewire of claim 103, where the first brace and the second brace comprise iron or aluminum

116. The guidewire of claim 103, where the first brace and the second brace comprise hardened polyurethane.

117. The guidewire of claim 103, where the first casing of the first brace, the second casing of the first brace, the first casing of the second brace and the second casing of the second brace each comprise a plastic such as hardened polyurethane.

118. The guidewire of claim 103, where the pivot is a screw comprising a head at one end and a distal end extending through matching holes in the center of the first brace and the center of the second brace, where the pivot comprises a transverse hole in the distal end to receive a pin, thereby fixing the pivot in place with respect to the first brace and the second brace.

119. The guidewire of claim 103, comprising one actuator base and associated piezoelectric strut.

120. The guidewire of claim 103, comprising a plurality of actuator bases and associated piezoelectric struts.

121. The guidewire of claim 103, comprising two actuator bases and associated piezoelectric struts.

122. The guidewire of claim 103, comprising three actuator bases and associated piezoelectric struts.

123. The guidewire of claim 103, comprising four actuator bases and associated piezoelectric struts.

124. The guidewire of claim 103, comprising more than four actuator bases and associated piezoelectric struts.

125. The guidewire of claim 103, where the guidewire comprises a plurality of actuator bases, each actuator base associated with one piezoelectric strut; and
where the actuator bases are spaced apart from each other longitudinally, and are rotated relative to one another axially, thereby causing the distal end of each piezoelectric strut to be connected to the inner surface of the outer layer in the distal section of the guidewire at a different location.

126. A system for performing a procedure within a human body, the system comprising:
a) one or more than one guidewire according to claim 103; and
b) a control connected to the guidewire for controlling the function of the guidewire.

127. The system of claim 126, where the guidewire further comprises wiring connecting the first piezoelectric core and the second piezoelectric core to the control for controlling transmission of current from the control to the first piezoelectric core and the second piezoelectric core.

128. The system of claim 126, where the guidewire further comprises wiring connecting the piezoelectric strut to the control for controlling transmission of current from the control to the piezoelectric strut.

129. The system of claim 126, where the guidewire comprises separate wiring connecting the first piezoelectric core and the second piezoelectric core of each actuator base to the control for controlling transmission of current from the control to the first piezoelectric core and the second piezoelectric core.

130. The system of claim 126, where the guidewire further comprises separate wiring connecting the piezoelectric strut associated with each actuator base to the control for controlling transmission of current from the control to the piezoelectric strut.

131. A method for performing a procedure within a human body, the method comprising:
a) providing a guidewire according to claim 103; and
b) using the guidewire to perform the procedure.

132. A method for performing a procedure within a human body, the method comprising:
a) providing a system according to claim 126; and
b) using the system to perform the procedure.

133. A method for performing a procedure within a human body, the method comprising:
a) providing a guidewire according to claim 103;
b) inserting the guidewire in a first configuration into an arterial lumen of the patient percutaneously;
c) supplying current to the one or more than one piezoelectric strut rendering the one or more than one piezoelectric strut in a first longitudinal length, and no current is being supplied to the one or more than one first piezoelectric core and the second piezoelectric core rendering them in a first longitudinal length, where the longitudinal axis of the distal section of the guidewire is coincident with the longitudinal axis of the intermediate section of the guidewire; and
d) in order to negotiate a curve of the arterial lumen, changing the guidewire into a second configuration by supplying current to the wiring to the one or more than one first piezoelectric core and the second piezoelectric core rendering them in a second longitudinal length and increasing the distance x, the axial separation of first piezoelectric core and the second piezoelectric core and decreasing the separation of the first casing of the first brace from the first casing of the second brace, and the separation of the second casing of the first brace from the second casing of the second brace, and by simultaneously ceasing to supply current to the wiring to the one or more than one piezoelectric strut rendering the one or more than one piezoelectric strut in a second longitudinal length;
where the first longitudinal length of the one or more than one first piezoelectric core and the second piezoelectric core is longer than the second longitudinal length of the one or more than one first piezoelectric core and the second piezoelectric core; and
where the first longitudinal length of the one or more than one piezoelectric strut is shorter than the second longitudinal length of the one or more than piezoelectric strut, thereby causing the longitudinal axis of the distal section of the guidewire to become non-coincident with the longitudinal axis of the intermediate section of the guidewire.

134. The method of claim 133, where the first longitudinal length of the one or more than one first piezoelectric core and the second piezoelectric core is longer than the second longitudinal length of the one or more than one first piezoelectric core and the second piezoelectric core by at least 25%.

135. The method of claim 133, where the first longitudinal length of the one or more than one first piezoelectric core and the second piezoelectric core is longer than the second longitudinal length of the one or more than one first piezoelectric core and the second piezoelectric core by at least 50%.

136. The method of claim 133, where the first longitudinal length of the one or more than one first piezoelectric core and the second piezoelectric core is longer than the second longitudinal length of the one or more than one first piezoelectric core and the second piezoelectric core by at least 100%.

137. The method of claim 133, where the second longitudinal length of the one or more than one piezoelectric strut is longer than the first longitudinal length of the one or more than piezoelectric strut by at least 25%.

138. The method of claim 133, where the second longitudinal length of the one or more than one piezoelectric strut is longer than the first longitudinal length of the one or more than piezoelectric strut by at least 50%.

139. The method of claim 133, where the second longitudinal length of the one or more than one piezoelectric strut is longer than the first longitudinal length of the one or more than piezoelectric strut by at least 100%.
